(19) United States
(12) Patent Application Publication
(10) Pub. No.: US 2008/0254121 A1
(43) Pub. Date: Oct. 16, 2008

(54) Multi-layer melatonin composition

(75) Inventors: Ken Clement, Oakville (CA); Shan Chaudhuri, Brampton (CA)

Correspondence Address: IOVATE HEALTH SCIENCE RESEARCH INC., 381 North Service Road West, Oakville, ON, L6M 0H4, CA

(73) Assignee: Iomedix Sleep International SRL, Belleville (BB)

(21) Appl. No.: 12/099,427

(22) Filed: Apr. 8, 2008

Related U.S. Application Data
(60) Provisional application No. 60/911,010, filed on Apr. 10, 2007.

(51) Int. Cl. A61K 9/24; A61K 31/4045; A61K 9/20; A61P 43/00

(52) U.S. Cl. 424/472; 514/415; 424/464

(57) ABSTRACT

A multi-layered solid dosage form for oral administration for a multi-phasic controlled release of Melatonin is described. The solid dosage form is useful as a composition to promote and maintain a state of sleep in an individual.

### RELATED APPLICATIONS

The present application is related to and claims benefit of priority to U.S. Provisional Patent Application Ser. No. 60/911,010, entitled “Mutli-layer melatonin composition,” filed Apr. 10, 2007, the disclosure of which is hereby fully incorporated by reference. Additionally, the instant application is related to the applicant's co-pending U.S. patent application Ser. No. 11/696,960 entitled “Composition for a Feeling of Relaxation”, Ser. No. 11/696,948 entitled “Composition for Supporting Restful Sleep”, and Ser. No. 11/696,945 entitled “Composition for a Feeling of Calmness”, all of which were filed on Apr. 5, 2007. The abovementioned co-pending U.S. Patent Applications are hereby fully incorporated by reference.

### FIELD OF THE INVENTION

The present invention relates to a multi-layered solid dosage form comprising Melatonin for oral administration for use as a sleep aid. In one aspect of the present invention, the arrangement of the individual layer is such that a temporally controlled and multi-phasic release of active ingredients results. In an additional aspect of the present invention the individual layers each provide distinct and differing dissolution characteristics to facilitate a temporally controlled and multi-phasic release of active ingredients.

### BACKGROUND OF THE INVENTION

Sleep occupies about one-third of our life and is necessary for mental and physical well-being. It additionally affects mood, behavior and physiology. Sleep and the control of sleep is a complex process involving multiple neuro-chemical pathways and associated brain structures. It is a dynamic process involving a shift in the balance of distinct physiological changes, involving both positive and negative neural signaling. The regulation of sleep in humans is governed by three processes—each influenced by hormonal and environmental factors: a daily sleep-wake cycle influenced by a circadian rhythm (24 hour cycle) tied to light-dark cycles.

The need for sleep is a biological drive similar to thirst or hunger. Interestingly though, the function of sleep is largely unknown, however some evidence indicates that sleep is required for learning. In North America, insomnia is estimated to affect a significant portion of the population every year and is associated with health problems and concomitant economic loss to society. It is clear that the impairment of sleep is detrimental to one's health. In humans, mild sleep deprivation results in indications of impaired immune system function. Prolonged sleep deprivation is even known to result in death. It has been determined by many that an individual can survive longer without food than one can without sleep; thus indicating the importance of sleep.

Strategies to improve sleep are beneficial, not only in terms of physical health, but also in terms of emotional health. Furthermore, reinforcement of sleep of adequate quantity and quality positively impacts most aspect of daily life.

### SUMMARY OF THE INVENTION

The foregoing needs and other needs and objectives that will become apparent for the following description are achieved in the present invention, which comprises Melatonin in a multi-layered solid dosage format for oral administration by an individual for the promotion and maintenance of sleep. In one embodiment, the individual layers are arranged such that a temporally controlled release of active ingredients results. In an additional embodiment, the individual layers each provide distinct and differing dissolution characteristics to facilitate a temporally controlled release of active ingredients.

### BRIEF DESCRIPTION OF DRAWINGS

FIG. 1A sagittal view of a caplet of an example embodiment.

FIG. 2 A sagittal view of a caplet of an example embodiment.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards multi-layered solid dosage forms comprising Melatonin for oral administration acting to promote and maintain a state of sleep in an individual. Advantageously, the use of a solid dosage form comprised of multiple, distinct layers with distinct properties or sequential arrangement, allows for the manipulation and control of the release of constituents contained within the various layers.

As used herein the term “unmodified-release” format is understood to be defined as pertaining to the dissolution and bioavailability profile of an ingested dietary ingredient wherein no additional modifications, be it chemical or physical, have been made to the ingredient with the specific intent to alter the dissolution or bioavailability profile from that of ingredient in a naturally occurring form. It is also understood that unmodified-release is, essentially, immediate-release of active ingredients. This is further understood to be a traditional- or conventional-release format where no slow-, delayed- or extended-release modifiers are therein incorporated.

As used herein the term “controlled-release” format is understood to be defined as a formulation and/or the physical arrangement of active ingredients and appropriate excipients in a specific format to facilitate a controlled- or non-immediate-release of active ingredients. The components of a controlled-release format may have been subjected to additional modifications, be it chemical or physical, with the specific intent to alter the dissolution or bioavailability profile from that of ingredient in a naturally occurring form.

As used herein the term “slow-release” format is understood to be defined as a controlled-release format wherein the release of active ingredients are delayed for a period of time or gradually released over an extended period of time. This is accomplished through the use of specific excipients and may include structural features designed to facilitate controlled-release. It is further understood that a slow-release format releases active ingredients at a rate slower than immediate-release.

As used herein the term “delayed-release” format is understood to be defined as a controlled-release format wherein the components of the delayed-release format have undergone specific modifications, be it physical or chemical, to facilitate the release of active ingredients at a specific time after ingestion. It is further understood that delayed-release formats, release active ingredients at a period of time later than unmodified release.

As used herein the term “quick-release” format is understood to be defined essentially as “unmodified-release”, as defined above. However, the term “quick-release” may further include components having modifications, chemical or physical, to enhance the rate of dissolution or bioavailability of active ingredients.

In all cases herein, the term “release” is relative to ingestion or administration by the individual as it is herein understood that the process of digestion instigates the dissolution of oral solid dosage forms such as the present invention.

Melatonin

Melatonin is a hormone produced by the pineal gland and is derived from the amino acid tryptophan. While possibly being involved in multiple biological processes, melatonin has largely been studied for its involvement in sleep regulation with respect to the circadian rhythm of an individual. Levels of melatonin cycle in the body based on lighting conditions—i.e. low melatonin levels during the day, higher levels at night. Typically, melatonin levels peak in the middle of the night and diminish thereafter. Melatonin has further been explored as a method to treat sleep disorders such as insomnia and ‘jet lag’ due to its apparent involvement the regulation of circadian rhythms. Melatonin supplementation in humans has been found to be efficacious for treating jet lag as well as hastening the onset of sleep.

In the preferred embodiment of the present invention, the solid dosage form is comprised of at least two distinct layers being positioned between an inner-most core and an outer-most coating. Each layer, the core and the coating contains an effective amount of Melatonin. The Melatonin will be released in a pre-determined manner according to the characteristics of the layer, the core or the coating as set forth below.

In the preferred embodiment the coating contains from about 1 mg to 5 mg of Melatonin available for an immediate release having a time period of about less than sixty-seconds. The preferred amount of Melatonin in the coating is about 1 mg. A portion of the Melatonin contained in the coating may be fine-milled to facilitate quick release. As used herein, the terms “fine-milled” and/or “fine-milling” refer the process of micronization. U.S. application Ser. No. 11/709,526 entitled “Method for Increasing the Rate and Consistency of Bioavailability of Supplemental Dietary Ingredients”, filed Feb. 21, 2007, which is herein fully incorporated by reference, discloses a method of improving the rate of bioavailability of compounds by increasing the rate of solubility. The increased rate of bioavailability of a compound or ingredients is achieved via a reduction in particle size using a “fine-milling” technique. Additionally, U.S. application Ser. No. 11/709,525 entitled “Method for a Supplemental Dietary Composition Having a Multi-Phase Dissolution Profile”, filed Feb. 21, 2007, which is herein fully incorporated by reference discloses a method of providing a multi-phasic dissolution profile of a composition. Any acceptable fine-milling technique will result in the fine-milled particles having an average particle size of between about 50 microns to about 2 microns. The reduction in size of the particle increases the surface area-to-volume ratio of each particle, thus increasing the rate of dissolution, thereby improving the rate of absorption.

As used herein, the terms “fine-milled” and/or “fine-milling” refer to the process of micronization. Micronization is a mechanical process which involves the application of force to a particle, thereby resulting in a reduction in the size of said particle.

As used herein, the term “particle size” refers to the diameter of the particle. The term “average particle size” means that at least 50% of the particles in a sample will have the specified particle size. Preferably, at least 80% of the particles in a given sample will have the specified particle size, and more preferably, at least 90% of the particles in a given sample will have the specified particle size.

As used herein, the term “solid dosage form” is defined as being a tablet or caplet or other means suitable to comprise a solid layered format wherein each layer has a different temporal dissolution profile, thus leading to the release of the active ingredients at specific time periods.

As used herein, the term “excipients” is defined as being any added material to the composition not considered to be an active component. Excipients may be used in the present invention as are commonly known in the art and may be employed for the purposes of fillers and binding agents. Excipients are also used herein to affect the dissolution profile of the individual components of the disclosed composition of the present invention. Examples of excipients which may be used in the present invention include, but are not limited to silicon dioxide, croscarmelose sodium, carboxymethyl cellulose, cross-linked povidone, starch, sodium starch glycolate, microcrystalline cellulose, hydropropylmethyl cellulose, and lactose. Other suitable excipients will be apparent to one of ordinary skill in the art.

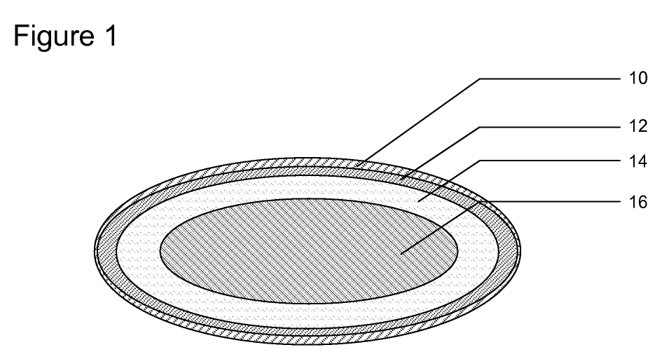

With reference to FIG. 1, the solid dosage form comprises a plurality of components sequentially arranged in layers from the outmost to the innermost of said solid dosage form; an outer coating 10, a first-layer 12, a second-layer 14 and an inner-core 16. Each of the layers containing a dosage of melatonin and excipients. The density of the first-layer 12, second-layer 14, and the inner core 16 are determined by the excipients and the compression applied during manufacturing to each of the layers 12, 14, and inner-core 16. The outer coating 10 as applied to the solid dosage form dissolves within 1 minute following oral administration, thus constituting an immediate-release profile. The first layer 12 begins to dissolve within about 60 seconds following administration to a mammal and is completely dissolved within about 2-hours following administration. The second-layer 14 begins to dissolve following the dissolution of the first-layer 12 at about 2-hours from the point of administration and is completely dissolved within about 4-hours following administration. The inner-core 16, begins to dissolve following the second-layer 14 at 4-hours following administration and is completely dissolved within about 6-hours following oral administration to a mammal. The interconnection of the dissolution profiles of the components of the present invention in this embodiment forms a multi-phase temporal release profile.

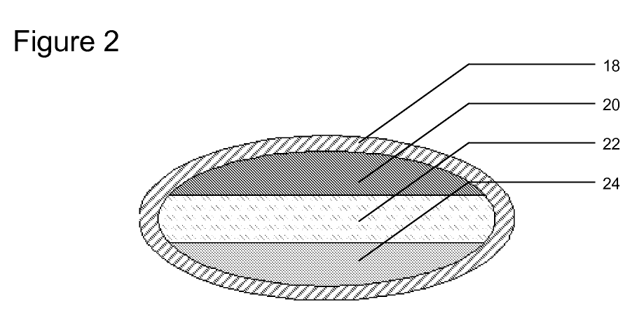

Another embodiment of the present invention is shown in FIG. 2. According to FIG. 2, an inner-core 22 is adjacently flanked to the opposite sides by a first-layer 20 and a second-layer 24, along the inner-core's 22, width, forming a solid dosage form 26. The first-layer 20 and the second-layer 24 are bonded to the inner-core 22 by the use of pharmaceutically acceptable binding agents suitable to maintain the aforementioned in contact as they are dissolved following ingestion by a mammal. The density of the first-layer 20, second-layer 24, and the inner core 22 are determined by the excipients and the compression applied during manufacturing to each of the layers 20, 24, and inner-core 22. Encompassing all the components, said first-layer 20, said second-layer 24 and said inner-core 22 is an outer-coating 18. The outer coating 18 dissolves within about 60 seconds following oral administration, thus constituting an immediate-release profile. The first layer 20 begins to dissolve within about 60 seconds, after the dissolution of the outer-coating 18, and is completely dissolved within about 2-hours following administration. The second-layer 24 begins to dissolve following the dissolution of the outer-coating 18, however the majority of said second-layer 24 dissolves from about 2-hours from the point of administration to said mammal and is completely dissolved within about 4-hours following administration. The inner-core 16, being adjacently flanked on opposite sides by said first-layer 20 and said second-layer 24 begins to dissolve following the dissolution of the outer-coating 18, however the majority the dissolution of said inner-corer 22 taking place about 4-hours from the point of administration and being completely dissolved within about 6-hours following administration. The interconnection of the dissolution profiles of the components of the present invention in this embodiment form a multi-phase temporal release profile.

According to one embodiment herein disclosed each of the outer-coating, first-layer, second-layer and inner-core contain melatonin. The outer-coating of the solid dosage form of the present invention comprises from about 0.1 mg to about 5 mg of melatonin. Preferably the outer coating comprises about 1 mg of melatonin which is released immediately following oral administration to a mammal. The first-layer of the solid dosage form of the present invention comprises from about 0.1 mg to about 5 mg of melatonin. Preferably, the first-layer comprises about 2 mg of melatonin to promote sleep as well as aid in maintaining sleep in a mammal during the period of 1 minute to 2-hours following oral administration to said mammal. The second-layer of the solid dosage form of the present invention comprises from about 0.1 mg to about 5.0 mg of melatonin. Preferably, the second layer comprises about 1.5 mg of melatonin to aid in maintaining sleep in a mammal during the period of 2-hours to 4-hours following oral administration to said mammal. The inner-core of the solid dosage form of the present invention comprises from about 0.1 mg to about 5.0 mg of melatonin. Preferably, the inner-core comprises about 0.5 mg of melatonin to aid in maintaining sleep in a mammal during the period of 4-hours to 6-hours following oral administration to said mammal.

The dissolution rate of the outer-coating, the first-layer, the second-layer and the inner-core, is determined by a combination of one or more excipients and the compression applied during manufacturing to each of the first-layer, second-layer and inner-core. The density of the layers, for the purposes of the present invention is achieved by varying the viscosity of the hydroxypropylmethyl cellulose used such that different dissolution profiles of each layer and the inner-core results. In the case of both embodiments presented herein, the inner-core 16 of FIG. 1 and the inner-core 24 of FIG. 2, the active ingredient, melatonin, binding agent excipients such as those selected from the group consisting of microcrystalline cellulose, hydroxypropylmethyl cellulose, and starch are compressed together at a force between 15 to 25 kN such that the majority of the dissolution occurs between 4-hours and 6-hours following oral administration. Furthermore, with reference to the second-layer 14 of FIG. 1 and the second-layer 24 of FIG. 2, the active ingredient, melatonin, and binding agent excipients such as those selected from the group consisting of microcrystalline cellulose, hydroxypropylmethyl cellulose, and starch are compressed together at a force between 15 to 25 kN such that the majority of the dissolution respective of said layer occurs between 2-hours and 4-hours following oral administration. Referring to the first-layer 12 of FIG. 1 and the first-layer 20 of FIG. 2, the active ingredient, melatonin, and binding agent excipients such as those selected from the group consisting of microcrystalline cellulose, hydroxypropylmethyl cellulose, and starch are compressed together at a force between 15 to 25 kN such that the majority of the dissolution occurs between 60-seconds and 2-hours following oral administration. In one embodiment, the different dissolution rates are achieved by sequential arrangement of layers from the outmost to the innermost of the solid dosage form. In an additional embodiment, the different dissolution rates are achieved in the present invention through the use of various excipients or erosion polymers combined with the degree of compression.

With reference to FIG. 2, said first-layer 20 is bonded to the inner-core 22 using one or more binding excipients in combination with compression sufficient for affixation as are known in the art. The resultantly bound first-layer 20 and inner-core 22 are bonded to said second-layer 24 using one or more binding excipients in combination with compression sufficient for affixation as is known in the art. The resultant multi-layered solid dosage 26 form is then coated with an outer-coating 18.

The present invention or those similarly envisioned by one of skill in the art may be utilized in methods to promote and maintain a state of sleep in an individual. As such, the present invention may be utilized as a sole method, or alternatively may be used in conjunction with other methods known to promote and maintain a state of sleep. Additionally, the present invention may incorporate additional ingredients known to promote and maintain a state of sleep.

In a preferred embodiment of the present invention, melatonin may be provided in a solid dosage form having specific controlled release characteristics. Advantageously, the composition may be provided in a layered solid dosage form. In such a form, each individual layer will provide unique dissolution characteristics. In this way a controlled release of the composition can be achieved. In one aspect of this embodiment, each layer contains a homogeneous mixture of ingredients whereby the release of all ingredients is dependent upon the characteristics of each given layer. In an alternative aspect of this embodiment, each layer contains a distinct set of specific ingredients which differ according to the layers such that different specific ingredients are released from the solid dosage form at different times according to a predetermined schedule. In all aspects of this embodiment, a temporally controlled release of ingredients is achieved.

It is herein understood that the immediate-release of Melatonin from the coating of the multi-layered solid dosage form of the present invention will promote the onset of a state of sleep. Additionally, it is herein understood that a further release of Melatonin up to about 2-hours will aid in the promotion of sleep. Furthermore, it is herein understood that the release of Melatonin, to a lesser degree than used to promote the onset of sleep from about 2-hours to about 4-hours will act to maintain a state of sleep. It is furthermore herein understood that the release of Melatonin throughout a period of about 4-hours to about 6-hours will act to further maintain a state of sleep.

The dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

Although the following examples illustrate the practice of the present invention in two of its embodiments, the examples should not be construed as limiting the scope of the invention. Other embodiments will be apparent to one of skill in the art from consideration of the specifications and example.

### EXAMPLES

### Example 1

A nutritional supplement to aid in achieving a restful night's sleep in the form of a caplet to be consumed before bedtime. The nutritional supplement consists of the following:
An outer-coating comprising about 3 mg fine-milled Melatonin for immediate release; a first-layer comprising about 0.5 mg Melatonin for slow-release up to about 45-minutes; a second-layer comprising about 0.5 mg Melatonin for delayed-release for about 2-hours; and an inner-core comprising about 1.0 mg Melatonin for delayed- and slow-release from about 4-hours to about 6-hours.

### Example 2

A nutritional supplement to aid in achieving a restful night's sleep in the form of a caplet to be consumed before bedtime. The nutritional supplement consists of the following:
An outer-coating comprising about 3 mg fine-milled Melatonin for immediate release; a first-layer comprising about 0.5 mg Melatonin, about 1.0 mg Catnip flower powder, about 1.0 mg Piscidia piscipula and about 25.0 mg deodorized Valerian root extract for slow-release up to about 45-minutes; a second-layer comprising about 0.5 mg Melatonin, about 75.0 mg deodorized Valerian root extract, 0.006 mg Methylcobalamin, about 5.0 mg Eclipta alba whole plant extract and about 1.0 mg Nardostachys jatamansi for delayed-release for about 2-hours; and an inner-core comprising about 1.0 mg Melatonin, about 100 mg Lemon balm extract and about 2.0 mg Mesua ferrea plant powder for delayed- and slow-release from about 4-hours to about 6-hours.

### Example 3

A nutritional supplement to aid in achieving a restful night's sleep in the form of a caplet to be consumed before bedtime. The nutritional supplement consists of the following:
An outer-coating comprising about 1.0 mg fine-milled Melatonin for immediate release; a first-layer comprising about 2.0 mg Melatonin for slow-release for up to about 2-hours after administration; a second-layer comprising about 1.5 mg Melatonin for delayed-release between about 2-hours and 4-hours after administration; and an inner-core comprising about 0.5 mg Melatonin for delayed- and slow-release between about 4-hours to about 6-hours after administration.

### Example 4

A nutritional supplement to aid in achieving a restful night's sleep in the form of a caplet to be consumed before bedtime. The nutritional supplement consists of the following:
An outer-coating comprising about 1.0 mg fine-milled Melatonin for immediate release; a first-layer comprising about 2.0 mg Melatonin, about 300 mg of lemon balm extract (Melissa officinalis), about 500 mg of Tryptophan, about 120 mg of Hops extract (Humulus iupulus), and about 50 mg of Griffonia simplicfolia for slow-release for up to about 2-hours after administration; a second-layer comprising about 1.5 mg Melatonin, about 2.0 mg of Mesua ferrea plant powder, and about 1.0 mg Nardostachys jatamansi for delayed-release between about 2-hours and 4-hours after administration; and an inner-core comprising about 0.5 mg Melatonin, about 30 mg of methylcobalamin and about 1.0 mg of Catnip flower powder (Nepeta cataria) for delayed- and slow-release between about 4-hours to about 6-hours after administration.

Extensions and Alternatives

In the foregoing specification, the invention has been described with a specific embodiment thereof, however, it will be evident that various modifications and changes may be made thereto without departing from the broader spirit and scope of the invention.

What is claimed:

1. A solid orally administrable dosage form comprising:
a first-layer comprising melatonin;
a second-layer comprising melatonin, wherein the second-layer is disposed immediately adjacent the first-layer; and
an inner-core comprising melatonin, wherein the inner-core is disposed immediately adjacent at least one of the group consisting of the first-layer and the second-layer.

2. The dosage form of claim 1, wherein the first-layer, second-layer and inner-core comprise different amounts of melatonin.

3. The dosage form of claim 1, wherein the inner-core is disposed immediately adjacent to the first-layer.

4. The dosage form of claim 1, wherein the inner-core us disposed immediately adjacent to the second-layer.

5. The dosage form of claim 1, wherein the inner-core is disposed immediately adjacent to each of the first-layer and the second-layer.

6. The dosage form of claim 1 further comprising an immediate release outer coating comprising fine-milled melatonin.

7. The dosage for of claim 6, wherein the outer coating comprises about 1.0 mg of fine-milled melatonin.

8. The dosage form of claim 1, wherein the first-layer comprises from about 0.1 mg to about 5.0 mg of melatonin.

9. The dosage form of claim 1, wherein the first-layer comprises about 2.0 mg of melatonin.

10. The dosage form of claim 1, wherein the second-layer comprises from about 0.1 mg to about 5.0 mg of melatonin.

11. The dosage form of claim 1, wherein the second-layer comprises about 1.5 mg of melatonin.

12. The dosage form of claim 1, wherein the inner-core comprises from about 0.1 mg to about 5.0 mg of melatonin.

13. The dosage form of claim 1, wherein the inner-core contains about 0.5 mg of melatonin.

14. A solid orally administrable dosage form comprising:
an inner-core adjacently flanked on opposite sides by a first-layer and a second-layer;
said inner-core comprises from about 0.1 mg and about 5 mg of melatonin in a delayed- and slow-release format;
said first-layer comprises from about 0.1 mg to about 5 mg of melatonin in a slow-release format; and
said second-layer comprises from about 0.1 mg to about 5 mg of melatonin in a delayed-release format.

15. A multilayer solid orally administrable dosage form, comprising:
an inner-core comprising from about 0.1 mg to about 5 mg of melatonin, which is sequentially over coated with a second-layer comprising from about 0.1 mg to about 5 mg of melatonin; and
a first-layer comprising from about 0.1 mg to about 5 mg of melatonin;
wherein the first-layer, second-layer and inner-core contain different amounts of melatonin.
